(19) United States
(12) Patent Application Publication
(10) Pub. No.: US 2008/0255193 A1
(43) Pub. Date: Oct. 16, 2008

(54) Pharmaceutical Composition

(76) Inventors: Jeffrey Brum, Collegeville, PA (US); Patrick G. Faulkner, King of Prussia, PA (US); Rennan Pan, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/088,661

(22) PCT Filed: Sep. 28, 2006
(86) PCT No.: PCT/US06/38406

§ 371 Date: Mar. 28, 2008

Related U.S. Application Data
(60) Provisional application No. 60/722,153, filed on Sep. 30, 2005.

(51) Int. Cl. A61K 31/47; A61P 11/00

(52) U.S. Cl. 514/312

(57) ABSTRACT

A novel pharmaceutical composition comprising the NK₃ receptor antagonist talnetant, povidone, mannitol and a surfactant, and a process for its preparation are disclosed.

The present invention relates to novel compositions containing the NK₃ receptor antagonist talnetant.

Talnetant, (S)-(−)-N-(α-ethylbenzyl)-3-hydroxy-2-phenylquinoline-4-carboxamide, (alternatively 3-hydroxy-2-phenyl-N-[(1S)-1-phenylpropyl]-4-quinolinecarboxamide), has the chemical structure (A).

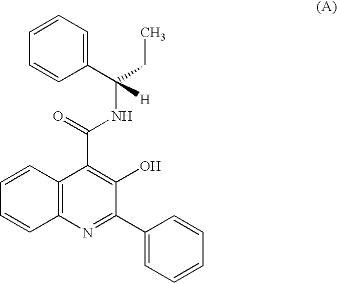

Talnetant, its preparation and its use in the treatment of pulmonary disorders, disorders of the central nervous system and neurodegenerative disorders are disclosed in published International Patent application WO 95/32948. Published International Patent applications WO 97/19927, WO 97/19928, WO 99/14196 and WO 02/094187 disclose additional therapeutic utilities for talnetant, pharmaceutically acceptable salts and processes for its preparation. WO 05/97077 discloses spray-dried compositions containing talnetant which have enhanced bioavalability. The above-mentioned patent applications are herein incorporated by reference as if each individual publication were specifically and individually indicated to be incorporated by reference herein as though fully set forth.

Talnetant has low aqueous solubility (approximately 0.03 mg/ml at pH 1 and 0.001 mg/ml, at pH 7.0). Typically drugs with low aqueous solubility are absorbed slowly across the walls of the gastrointestinal tract (GIT) due to poor dissolution of the solid in the GIT leading to a small diffusive driving force.

There are a number of different methods employed to improve absorption of a particular drug substance. It may be possible to develop so-called prodrugs or salts of the active agent, i.e. more soluble derivatives, by attaching a solubilizing group (e.g. phosphate, succinate or polyethylene glycol) to the drug, thereby taking advantage of the high solubility and dissolution rate of the derivative prodrug/salt. Alternatively, it is known to use physical formulation methods, such as use of amorphous drug or dispersion in a soluble carrier to increase the dissolution rate of the drug product and hence the absorption rate (J. H. Fincher, J. Pharm. Sci., 1968, 57, 1825 and G. L. Amidon et al., J. Pharm. Sci., 1980, 12, 1363).

A further alternative is to decrease the particle size of the drug. Decreasing the particle size increases the surface area of the drug particle, thereby increasing its dissolution rate.

A variety of processes have been developed to prepare fine particles of drug substance. Typically, dry milling techniques are used for the preparation of particulate medicaments (see E. L. Parrott, J. Pharm. Sci., 1974, 63, 813). Air jet milling and fluid energy milling (micronising) have been favoured because of the reduced risk of introducing contamination from mill materials. More recently, particles having a size of less than 1 μm have been obtained using wet milling processes. For example, published European Patent application EP-A-0 262 560 describes the use of wet milling techniques to prepare compositions containing benzoyl urea derivatives in which the average particle size is 1 μm or less. Provision of the fine particles is said to improve the absorbability from the GIT of the poorly water soluble benzoyl urea compounds thereby increasing their bioavailability. European Patent application EP-0 499 299 B1 describes a wet milling procedure for preparing particles of a crystalline drug substance, which particles have a surface modifier adsorbed on the surface in an amount sufficient to maintain an effective average particle size of less than about 400 nm.

Aqueous dispersions obtained from wet milling processes may be used directly as a therapeutic agent if prepared under conditions of appropriate hygiene, for example, by using water and other components which meet Ph Eur/USP standards. For the preparation of formulations for use in human therapy, it is preferred that the aqueous dispersion is converted to a dry powder. This is suitably carried out by spray drying the resulting aqueous dispersion, typically collecting the product from the dryer using a cyclone separator. The resulting aqueous dispersion may also be spray granulated.

The objective of spray drying is to remove water from dispersions of drug particles so that the powder can be processed further to prepare capsule or tablet or other suitable oral dosage form. However, it is desirable that particles obtained from the spray dried powder are substantially the same size when dispersed in aqueous medium as the freshly-milled particles. If particles of the same size as the freshly-milled particles are obtained, it is referred to in the art (and hereinafter) as complete “recovery of particle size”.

However, spray drying certain wet-milled dispersions of talnetant results in a poor recovery of particle size, i.e. a significant increase in particle size is seen when the spray dried particles are added to aqueous media. Spray drying wet-milled dispersions of talnetant containing certain excipients may address this problem, and result in increased recovery of particle size and bioavailability. However, this may not necessarily address certain processing issues, particularly picking or sticking during tabletting if the spray dried powder is compressed into tablets.

In the preparation of drug tablets on a commercial scale, spray dried powder may be prepared and mixed with external excipients to produce a compression blend. The blend may then be compressed into tablets, which may subsequently be coated with a coating such as an aqueous film coating. For the tabletting of a compression blend containing a spray dried powder, common problems such as sticking/picking/filming may be encountered. Sticking is a phenomenon of the tablet surface sticking to the punch face. Picking is a form of sticking in which a small portion of granulation sticks to the punch face and grows over time, picking out a cavity on the tablet face. Filming is a slow form of picking (for more details, see for example H. Lieberman, L. Lachman, and J. Schwartz, “Pharmaceutical Dosage Forms: Tablets”, Volume 1, Second Edition, 1989). If the compaction properties of a compression blend of a drug is poor, especially if tablet sticking/picking is present, tabletting on a commercial scale is virtually impossible. Running a tablet press at a slow speed, control of the relative humidity of the environment, and/or use of special microfinished tooling may alleviate the picking/sticking problem. However, this problem can not be eliminated.

For the commercial manufacture of talnetant tablets, it is important that the formulation has good compaction properties as well as maintaining bioavailability. We have previously shown (WO 05/07077) that spray drying wet-milled dispersions of talnetant containing one of more ionic surfactant, povidone, and one or more soluble carrier, such as mannitol, results in talnetant compositions with good bioavailability. It has now been found that formulations with good compaction properties and good bioavailability may be achieved by manipulating the ratios of talnetant, mannitol and povidone.

The present invention provides a pharmaceutical composition comprising (i) talnetant, (ii) povidone, (iii) mannitol and (iv) a surfactant, wherein:
(a) the ratio of povidone to mannitol is 0.45:1 or higher; and
(b) the ratio of (povidone+mannitol) to talnetant is 0.3:1 or higher.

Unless otherwise stated, all ratios herein refer to weight to weight (w/w).

In one embodiment, the ratio of povidone to mannitol is between 0.45:1 and 1:1. In one embodiment, the ratio of povidone to mannitol is around 0.5:1, for example between 0.45:1 and 0.55:1, or for example 0.45:1 or 0.5:1. In one embodiment, the ratio of povidone to mannitol is around 0.6:1, for example between 0.58:1 and 0.62:1, or for example 0.59:1 or 0.58:1.

In one embodiment, the ratio of (mannitol+povidone) to talnetant is between 0.3:1 and 5:1. In one embodiment, the ratio of (mannitol+povidone) to talnetant is between 0.6:1 and 1:1. In one embodiment, the ratio of (mannitol+povidone) to talnetant is around 0.5:1, for example between 0.45:1 and 0.55:1, or for example 0.54:1. In one embodiment, the ratio of (mannitol+povidone) to talnetant is around 0.75:1, for example between 0.7:1 and 0.8:1, or for example 0.74:1 or 0.76:1.

In one embodiment, talnetant is in the form of talnetant particles having a Dv90 in the range from 0.1 to 2.0 μm. In another embodiment, talnetant is in the form of talnetant particles having a Dv90 in the range from 0.2 to 0.5 μm. As used herein, the term Dv90 refers to the value in micrometers at the 90^{th} percentile of a volume distribution derived from low angle laser light scattering (Malvern Mastersizer 2000). Similarly the Dv50 and Dv10 parameters refer to the 50^{th} and 10^{th} percentiles respectively of the same distribution.

To obtain talnetant particles having a Dv90 in the range from 0.1 to 2.0 μm, or from 0.2 to 0.5 μm, talnetant may be first wet-milled in any suitable aqueous, non-aqueous or organic solvent (e.g. an oil), and then spray dried. Suitable milling apparatus include conventional wet bead mills such as those manufactured by Nylacast (available from Nylacast Components, 200 Hastings Road, Leicester, LE5 0HL, UK), Netzsch (available from Erich NETZSCH GmbH & Co. Holding KG Gebrüder-Netzsch-StraBe 19, D-95100 Selb, Germany), Drais (available from Draiswerke, Inc, 40 Whitney Road, Mahwah, N.J. 07430, USA) and others. The milling chamber of the milling apparatus may be lined with or constructed from an abrasion-resistant polymer material. The milling chamber of the milling apparatus may be lined with or constructed from nylon. An example of a suitable milling chamber is described in International Patent Publication WO 02/00196. Suitable grinding media include glass beads and ceramic beads, for example, those made from rare earth oxide materials. The diameter of said grinding media is for example within the range 0.1 mm to 3 mm, suitably within the range 0.3 mm to 0.8 mm. The density of said grinding media is for example greater than 3 gcm⁻³, suitably within the range 5 to 10 gcm⁻³. Suitable spray drying and spray granulating techniques will be apparent to those skilled in the art (see for example, Gilbert S. Banker, “Modern Pharmaceutics, Drugs and the Pharmaceutical Sciences”, 1996 and references cited therein) and may be effected using a spray dryer, such as the Niro SD 6.3R Spray Dryer (Niro A/S, Gladsaxevej 305, 2860 Soeborg, Denmark), the Niro Mobile Minor, the Yamato GA-32 Spray Dryer (2-1-6 Nihonbashi Honcho, Chuo-ku, Tokyo, 103-8432, Japan) or a fluid bed granulator, such as the Glaft fluid bed granulator. Talnetant particles may be sized using conventional techniques known in the art, such as laser light diffraction and photon correlation spectroscopy.

Povidone (also known as polyvinyl pyrolidone or PVP) is an anti-agglomeration agent. Examples include Kollidon 30 and Plasdone K29/32.

Mannitol is a soluble carbohydrate which acts as a soluble carrier. Suitably, mannitol powder is used (for example Mannitol 60). Mannitol exists in three crystalline polymorphic forms: α, β and δ (see for example Burger, A., Henc, J. Rollinger, J., Weissnicht, A., Stottner, H. J. Pharm Sci, 89, 457, (2000)). XPRD and DSC analyses of a spray dried composition of the present invention indicates that, in the compositions of the present invention, all of the crystalline mannitol exists in the δ polymorphic form. However, it should be understood that there may also be a small amount of amorphous mannitol present in the spray dried composition. When the ratio of povidone:mannitol is lower than 0.45:1, XRPD and DSC indicate that crystalline mannitol exists in a mixture of the α, δ polymorphs with no clear evidence of the β form. It is therefore believed that the presence of the majority of the crystalline mannitol in the β form is important in solving the sticking and picking problem (see for example Yoshinari T., Forbes, R., York, P., and Kawashima, Y., International Journal of Pharmaceutics 258, 121-131 (2003)). The mannitol may be added to the dispersion prior to wet milling. Alternatively, it may be added to the wet milled dispersion before spray drying.

The surfactant in the composition of the present invention may be an ionic surfactant or a non-ionic surfactant. If an ionic surfactant is used, it may be an anionic surfactant or a cationic surfactant. Examples of anionic surfactants include alkyl sulfates such as sodium lauryl sulfate, and dioctyl sodium sulfosuccinate (docusate sodium. Examples of cationic surfactants include cetyl pyridinium chloride and cetyl trimethylammonium bromide. In an embodiment, the surfactant is an anionic surfactant. In a further embodiment, the surfactant is sodium lauryl sulfate or dioctyl sodium sulfosuccinate (docusate sodium). In a still further embodiment the surfactant is sodium lauryl sulfate.

In one embodiment, the surfactant is a non-ionic surfactant. Examples of non-ionic surfactants include POE alkylphenols, POE straight-chain alcohols, POE polyoxypropylene glycols, POE mercaptans, long-chain carboxylic acid esters such as glyceryl and polyglyceryl esters of natural fatty acids, propylene glycol, sorbitol and POE sorbitol esters, polyoxyethylene glycol esters etc. In a further embodiment, the non-ionic surfactant is a POE polyoxypropylene glycol.

In one embodiment, the concentration of surfactant in the spray dried composition is 0.05 to 50.0% by weight of talnetant. In another embodiment, the concentration of surfactant in the dispersion prior to spray drying is 0.05 to 10.0% by weight of dispersion, in a further embodiment the concentration of surfactant in the dispersion prior to spray drying is 0.05 to 2.0% by weight of dispersion.

In one embodiment, the dispersion contains 0.001 to 0.1 moles of ionic surfactant per mole of talnetant. In a further embodiment, the dispersion contains 0.005 to 0.05 moles of surfactant per mole of talnetant.

The composition of the present invention may contain further suitable pharmaceutically acceptable excipients, which may be added. Suitable excipients are described in the Handbook of Pharmaceutical Excipients, Pharmaceutical Press, 5^{th} Edition, 2006 published by The American Pharmaceutical Association and The Royal Pharmaceutical Society of Great Britain. Examples of further excipients include stablilisers to maintain the particles in suspension.

The composition of the present invention may be subjected to dry milling, wet milling and/or spray drying. As discussed above, for wet milling, typically water is used as an aqueous medium, and then removed by spray drying to obtain a spray dried powder. Thus in one embodiment, the composition of the present invention also comprises water. In one embodiment, the composition of the present invention comprises 25% to 90% water by unit formula % w/w. In one embodiment, the composition of the present invention consists of:

| Ingredients | Unit Formula % w/w |
|---|---|
| Talnetant | 20.0 |
| Sodium Lauryl Sulfate | 0.3 |
| Povidone | 5.5 |
| Mannitol | 9.5 |
| Water | 64.7 |

In another aspect, there is provided a process for the preparation of a spray-dried composition, the process comprising:

(1) wet milling a dispersion of (i) talnetant, (ii) povidone, (iii) mannitol and (iv) a surfactant, wherein:
(a) the ratio of povidone to mannitol is 0.45:1 or higher; and
(b) the ratio of (povidone+mannitol) to talnetant is 0.3:1 or higher.
   (2) spray drying or spray granulating the resulting dispersion.

In another aspect, the present invention provides a spray dried composition obtainable according to the process as defined above. Thus the present invention provides a spray dried composition comprising (i) talnetant, (ii) povidone, (iii) mannitol and (iv) a surfactant, wherein:
(a) the ratio of povidone to mannitol is 0.45:1 or higher; and
(b) the ratio of (povidone+mannitol) to talnetant is 0.3:1 or higher.

The composition of the present invention may be administered to the subject without further processing. However, it will generally be formulated into other dosage forms in conjunction with further pharmaceutically acceptable excipients selected with regard to the desired dosage form. These further excipients will typically be added to the spray dried composition after spray drying.

Thus in one embodiment, there is provided a dosage form comprising a composition defined in the first aspect.

In one embodiment, the dosage form is administered orally. Oral administration will typically involve swallowing so that the compound enters the GIT. Dosage forms for oral administration include solid formulations such as tablets, capsules (containing particulates, powders or non-aqueous suspension), sachets, vials, powders, granules, lozenges, reconstitutable powders and liquid preparations (such as suspensions, emulsions and elixirs).

Oral dosage forms may contain further excipients such as binding agents (for example syrup, acacia, gelatin, sorbitol, starch, PVP, HPMC, and tragacanth); fillers (for example lactose, sugar, maize-starch, calcium phosphate, sorbitol and glycine); tabletting lubricants (for example magnesium stearate); glidants (for example colloidal silicon dioxide such as Cab-O-Sil M-5P) and disintegrants [for example starch, crospovidone (Polyplasdone XL), croscarmellose sodium, sodium starch glycolate and microcrystalline cellulose (Avicel PH 102)]. In addition, the oral dosage form may contain preservatives, anti-oxidant, flavours, granulation binders, wetting agents and colorants.

In an embodiment, the dosage form for oral administration is a tablet. Tablets may be prepared using standard technology familiar to the formulation scientist, for example by direct compression, granulation, melt congealing and extrusion. The tablet may be coated or uncoated. The tablet may be formulated to be immediate or controlled release. Controlled release formulations include delayed-, sustained-, pulsed- or dual-release. Suitable tabletting excipients are described in the Handbook of Pharmaceutical Excipients, Pharmaceutical Press, 5^{th} Edition, 2006 published by The American Pharmaceutical Association and The Royal Pharmaceutical Society of Great Britain. Typical tabletting excipients include: carriers [for example microcrystalline cellulose (Avicel PH 102)], lubricating agents (for example magnesium stearate), binding agents, wetting agents, colorants, flavourings, glidants [for example Colloidal Silicon Dioxide (Cab-O-Sil M-5 P)] and disintegrants [for example crospovidone (Polyplasdone XL)].

In one embodiment, the dosage form consists of:

| Ingredients | Unit Formula % w/w |
|---|---|
| Talnetant | 38.46 |
| Sodium Lauryl Sulfate | 0.58 |
| Povidone | 10.58 |
| Mannitol Powder | 18.27 |
| Excipient(s) | to make up 100% |

In one embodiment, the dosage form consists of:

| Ingredients | Unit Formula % w/w |
|---|---|
| Talnetant Spray Dried Powder | 67.9 |
| consisting of: | |
| Talnetant drug substance | 38.46 |
| Sodium Lauryl Sulfate | 0.58 |
| Povidone (Kollidon 30) | 10.58 |
| Mannitol | 18.27 |
| Microcrystalline Cellulose | 20.6 |
| Colloidal Silicon Dioxide | 0.5 |
| Crospovidone | 10.0 |
| Magnesium Stearate | 1.00 |
| Total | 100.0 |

Excipients suitable for preparing liquid dosage forms include: suspending agents (for example sorbitol, syrup, methyl cellulose, gelatin, hydroxyethylcellulose, carboxymethylcellulose, aluminium stearate gel and hydrogenated edible fats); emulsifying agents (for example lecithin, sorbitan monooleate and acacia); aqueous or non-aqueous vehicles, which include edible oils (for example almond oil and fractionated coconut oil), oily esters (for example esters of glycerine and propylene glycol), ethyl alcohol, glycerine, water and normal saline; preservatives (for example methyl, propyl p-hydroxybenzoate and sorbic acid); and if desired conventional flavouring or colouring agents.

The effective dose of talnetant depends on the condition of the patient, the frequency and route of administration. A unit dose will generally contain from 20 to 1000 mg of talnetant, in an embodiment 30 to 800 mg, in a further embodiment 200 to 600 mg. The unit dose may be administered one or more times per day (for example 2, 3 or 4 times per day). The total daily dose for a 70 kg adult will normally be in the range 100 to 3000 mg. Alternatively the unit dose will contain from 2 to 20 mg of active ingredient and be administered in multiples, if desired, to give the preceding daily dose.

In an embodiment, the compositions and tablets of the invention are adapted for use in the medical or veterinarial fields. For example, such preparations may be in a pack form accompanied by written or printed instructions for use as an agent in the treatment of the conditions.

NK₃ receptor antagonists, including talnetant, are useful in the treatment and prevention of a wide variety of clinical diseases and conditions characterised by overstimulation of the NK₃ receptors. These diseases and conditions (hereinafter referred to as “diseases and conditions of the invention”) include: CNS disorders such as depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); anxiety disorders (including generalised anxiety disorder, social anxiety disorder, agitation, tension, social or emotional withdrawal in psychotic patients, panic disorder, and obsessive compulsive disorder); phobias (including agoraphobia and social phobia); psychosis and psychotic disorders (including schizophrenia, schizo-affective disorder, schizophreniform diseases, acute psychosis, alcohol psychosis, autism, delerium, mania (including acute mania), manic depressive psychosis, hallucination, endogenous psychosis, organic psychosyndrome, bipolar disorder including subconditions associated with bipolar disorder as listed in Diagnostic and Statistical Manual of Mental Disorders, 4^{th} Edition, published by the American Psychiatric Association (DSM-IV), paranoid and delusional disorders, puerperal psychosis, and psychosis associated with neurodegenerative diseases such as Alzheimer's diease); post-traumatic stress disorder; attention deficit hyperactive disorder; cognitive impairment (e.g. the treatment of impairment of cognitive functions including attention, orientation, memory (memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states)); convulsive disorders such as epilepsy (which includes simple partial seizures, complex partial seizures, secondary generalised seizures, generalised seizures including absence seizures, myoclonic seizures, clonic seizures, tonic seizures, tonic clonic seizures and atonic seizures); psychosexual dysfunction (including inhibited sexual desire (low libido), inhibited sexual arousal or excitement, orgasm dysfunction, inhibited female orgasm and inhibited male orgasm, hypoactive sexual desire disorder, female sexual desire disorder, and sexual dysfunction side-effects induced by treatment with antidepressants of the SSRI-class); sleep disorders (including disturbances of circadian rhythm, dyssomnia, insomnia, sleep apnea and narcolepsy); disorders of eating behaviours (including anorexia nervosa and bulimia nervosa); neurodegenerative diseases (such as alzheimer's disease, amyotropic lateral sclerosis, motor neuron disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); withdrawal from abuse of drugs including smoking cessation or reduction in level or frequency of such activities (such as abuse of cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine, methylamphetamine or a combination thereof); pain (which includes neuropathic pain (including diabetic neuropathy; sciatica; non-specific lower back pain; multiple sclerosis pain; pain associated with fibromyalgia or cancer; AIDS-related and HIV-related neuropathy; chemotherapy-induced neuropathy; neuralgia, such as post-herpetic neuralgia and trigeminal neuralgia; sympathetically maintained pain and pain resulting from physical trauma, amputation, cancer, toxins or chronic inflammatory conditions such as rheumatoid arthritis and osteoarthritis; reflex sympathetic dystrophy such as shoulder/hand syndrome), acute pain (e.g. musculoskeletal pain, post operative pain and surgical pain), inflammatory pain and chronic pain, pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia), pain associated with migrane, and non-cardiac chest pain); certain CNS-mediated disorders (such as emesis, irritable bowel syndrome and non-ulcer dyspepsia); and pulmonary disorders (such as asthma, chronic obstructive pulmonary disease, airway hyperreactivity and cough).

More diseases or conditions (hereinafter referred to as “preferred diseases and conditions of the invention”) mediated by modulation of the NK₃ receptor include depression; anxiety disorders; phobias; psychosis and psychotic disorders; post-traumatic stress disorder; attention deficit hyperactive disorder; withdrawal from abuse of drugs including smoking cessation or reduction in level or frequency of such activities; irritable bowel syndrome; cognitive impairment; convulsive disorders; psychosexual dysfunction; sleep disorders; disorders of eating behaviours; neurodegenerative diseases; pain; emesis; irritable bowel syndrome; non-ulcer dyspepsia; and pulmonary disorders (such as asthma, chronic obstructive pulmonary disease, airway hyperreactivity and cough).

The following Examples illustrate the present invention.

### Model for Evaluation of Sticking/Picking on Tabletting

A compression model was developed for evaluation of tablet sticking/picking. This model is as follows:
Manesty Beta press running at 40 RPM.
4 non-microfinished standard round concave punches.
Relative humidity controlled at 50%.
Compression for 20-30 minutes at 2 kg batch size
Visual observation of punch surfaces after tabletting.

### Analytical Methods

Particle size distribution of nanomilled suspensions may be directly measured on a Malvern Mastersizer 2000, a Laser Light Diffraction instrument. For the particle size recovery of spray dried powders, a spray dried powder is gently mixed with water (50 mg in 10 mL of water), and the resulting suspension is used for the particle size measurement. For the evaluation of particle size recovery of tablets, the following procedure may be used:
Add a tablet into water and allow it to disintegrate.
Filter the resultant slurry to remove the excipients.
Measure the particle size distribution of the filtrate.
Measure the assay of the filtrate.
Calculate the particle size recovery of the tablet.

Particle size recovery of tablets is the percentage of the drug particles re-dispersed back to submicron size.

### EXAMPLE 1

### Comparative Example

Compositions 1 and 2

| | Composition 1 | |
|---|---|---|
| | (unit formula | Composition 2 |
| Ingredients | % w/w) | (unit formula % w/w) |
| Talnetant, micronised | 20.0 | 20.0 |
| Sodium Lauryl Sulfate | 0.300 | 0.300 |
| Povidone (Kollidon 30) | 1.70 | 1.70 |
| Mannitol Powder (Mannitol 60) | 5.00 | 10.00 |
| Purified Water | 73.0 | 68.0 |
| Total | 100.0 | 100.0 |
| Ratio of povidone:mannitol | 0.34 | 0.17 |
| Ratio of | 0.34 | 0.59 |
| (povidone + mannitol):talnetant | | |

For composition 1, sodium lauryl sulfate was dissolved in the water. Talnetant (Dv90 approximately 20 to 30 μm) and povidone were then added with continuous mixing until a uniform suspension was obtained. Mannitol powder USP was then added to the dispersion until a uniform suspension was obtained. The homogenous suspension was passed through a Netzsch bead mill (containing 85% by volume of yttrium-stabilised 0.3 mm zirconium oxide beads). The dispersion was re-circulated through the bead mill with continual mixing until a target particle size Dv90 of about 0.4 μm was obtained. The suspension was spray-dried using a Niro Mobile Minor spray dryer (operated in accordance with the manufacturers instructions) at the following settings: two-fluid nozzle: 2 bar atomisation pressure; drying gas flowrate: 65 m³/hr; suspension spray rate: around 35 to 50 g/min; inlet temperature: around 150° C.; outlet temperature: around 60° C. The spray dried powder was then mixed with further excipients as follows:

| | Composition 1 |
|---|---|
| | Unit formula |
| Ingredients | (mg/tablet) |
| Talnetant spray dried powder, 74.1% w/w | 270.0 |
| consisting of: | |
| Talnetant, micronised | 200.0 |
| Sodium Lauryl Sulfate | 3.00 |
| Povidone (Kollidon 30) | 17.0 |
| Mannitol | 50.0 |
| Microcrystalline Cellulose (Avicel | 195.0 |
| PH102) | |
| Crospovidone (Polyplasdone XL) | 30.0 |
| Magnesium stearate | 5.00 |
| Total | 500.0 |

The compression blend thus obtained was then tested by the tabletting model described above.

A compression blend for composition 2 was prepared and tested in a similar manner to Composition 1, except that in the preparation, after dissolving the sodium lauryl sulfate in water, talnetant, povidone and mannitol were added together and mixed. The resulting homogenous suspension was passed through a Netzsch bead mill (containing 85% by volume of yttrium-stabilised 0.3 mm zirconium oxide beads). The dispersion was re-circulated through the bead mill with continual mixing until a target particle size Dv90 of about 0.45 μm was obtained.

| | Composition 2 |
|---|---|
| | Unit formula |
| Ingredients | (mg/tablet) |
| Talnetant spray dried powder, 64% w/w | 270.0 |
| consisting of: | |
| Talnetant, micronised | 200.0 |
| Sodium Lauryl Sulfate | 3.00 |
| Povidone (Kollidon 30) | 17.0 |
| Mannitol | 100.0 |
| Colloidal Silicon Dioxide | 5.00 |
| (Cab-O-Sil M-5 P) | |
| Microcrystalline Cellulose (Avicel PH102) | 140.0 |
| Crospovidone (Polyplasdone XL) | 30.0 |
| Magnesium stearate | 5.00 |
| Total | 500.0 |

For both compositions, severe sticking and picking were observed after 5 minutes of compression and at the end of the compression run. For Composition 1, the particle size recovery of the resulting spray dried powder is nearly complete. For Composition 2, the particle size recovery of the resulting spray dried powder is complete.

By XRPD and DSC, the mannitol in both spray dried powders exists as a mixture of α form and δ form.

### EXAMPLE 2

### According to the Invention

### Composition 3

The following composition was prepared in a similar manner to Composition 1 in Example 1, except that, after dissolving the sodium lauryl sulfate in water, talnetant, povidone and mannitol were added together and mixed. The resulting homogenous suspension was passed through a Netzsch bead mill (containing 85% by volume of yttrium-stabilised 0.3 mm zirconium oxide beads). The dispersion was re-circulated through the bead mill with continual mixing until a target particle size Dv90 of about 0.45 μm was obtained.

| Ingredients | Unit Formula % w/w |
|---|---|
| Talnetant, micronised | 20.0 |
| Sodium Lauryl Sulfate | 0.3 |
| Povidone (Kollidon 30) | 5.5 |
| Mannitol Powder (Mannitol 60) | 9.5 |
| Purified Water | 64.7 |
| Total | 100.0 |
| Ratio of povidone:mannitol | 0.58 |
| Ratio of (povidone + mannitol):talnetant | 0.75 |

The above composition was spray dried in a similar manner to Example 1. Spray drying was conducted on a Niro Mobile Minor spray dryer at the inlet/outlet temperatures of about 150°/60° C. Batch size was 10 kg of suspension. The spray dried powder was then mixed with further excipients as follows:

| | Unit formula |
|---|---|
| Ingredients | (mg/tablet) |
| Talnetant spray dried powder 56.7% w/w | 353.0 |
| consisting of: | |
| Talnetant, micronised | 200.0 |
| Sodium Lauryl Sulfate | 3.00 |
| Povidone (Kollidon 30) | 55.0 |
| Mannitol | 95.0 |
| Colloidal silicon dioxide (Cab-O-Sil M-5 P) | 2.60 |
| Microcrystalline Cellulose (Avicel PH102) | 107.2 |
| Crospovidone (Polyplasdone XL) | 52.0 |
| Magnesium stearate | 5.20 |
| Total | 520.0 |

No sticking was observed during compression. Furthermore, particle size recovery of the resulting tablets was greater than 90%, while the particle size recovery of the resulting spray dried powder was complete.

By XRPD and DSC, the mannitol in the spray dried powder exists as δ form.

### EXAMPLE 3

### According to the Invention

### Composition 4

The following composition was prepared in a similar manner to Composition 3 in Example 2. The homogenous suspension was passed through a Netzsch bead mill (containing 85% by volume of yttrium-stabilised 0.3 mm zirconium oxide beads). The dispersion was re-circulated through the bead mill with continual mixing until a target particle size Dv90 of about 0.45 μm was obtained.

| Ingredients | Unit Formula % w/w |
|---|---|
| Talnetant, mioronised | 20.0 |
| Sodium Lauryl Sulfate | 0.300 |
| Povidone (Kollidon 30) | 5.00 |
| Mannitol Powder (Mannitol 60) | 5.00 |
| Purified Water | 69.7 |
| Total | 100.0 |
| Ratio of povidone:mannitol | 1 |
| Ratio of (povidone + mannitol):talnetant | 0.5 |

The composition was spray dried in a similar manner to Example 1. Spray drying was conducted on a Niro Mobile Minor spray dryer at the inlet/outlet temperatures of about 150°/60° C. The spray dried powder was then mixed with further excipients as follows:

| | Unit formula |
|---|---|
| Ingredients | (mg/tablet) |
| Talnetant spray dried powder, 66.0% w/w | 303 |
| consisting of: | |
| Talnetant, micronised | 200.0 |
| Sodium Lauryl Sulfate | 3.00 |
| Povidone (Kollidon 30) | 50.0 |
| Mannitol | 50.0 |
| Colloidal silicon dioxide (Cab-O-Sil M-5 P) | 5.00 |
| Microcrystalline Cellulose (Avicel PH102) | 157 |
| Crospovidone (Polyplasdone XL) | 30.0 |
| Magnesium stearate | 5.00 |
| Total | 500.0 |

No sticking was observed during compression. The particle size recovery of the resulting spray dried powder was complete.

By XRPD and DSC, the mannitol in the spray dried powder exists as δ form.

### EXAMPLE 4

### According to the Invention

### Composition 5

The following composition was prepared in a similar manner to Composition 3 in Example 2. The homogenous suspension was passed through a Netzsch bead mill (containing 85% by volume of yttrium-stabilised 0.3 mm zirconium oxide beads). The dispersion was re-circulated through the bead mill with continual mixing until a target particle size Dv90 of about 0.45 μm was obtained.

| Ingredients | Unit Formula % w/w |
|---|---|
| Talnetant, micronised | 20.0 |
| Sodium Lauryl Sulfate | 0.300 |
| Povidone (Kollidon 30) | 3.35 |
| Mannitol Powder (Mannitol 60) | 7.50 |
| Purified Water | 68.85 |
| Total | 100.0 |
| Ratio of povidone:mannitol | 0.45 |
| Ratio of | 0.54 |
| (povidone + mannitol):talnetant | |

The composition was spray dried in a similar manner to Example 1. Spray drying was conducted on a Niro Mobile Minor spray dryer at the inlet/outlet temperatures of about 150°/60° C. The spray dried powder was then mixed with further excipients as follows:

| Ingredients | Unit formula (mg/tablet) |
|---|---|
| Talnetant spray dried powder, 64.2% w/w | 311.5 |
| consisting of: | |
| Talnetant, micronised | 200.0 |
| Sodium Lauryl Sulfate | 3.00 |
| Povidone (Kollidon 30) | 33.5 |
| Mannitol | 75.0 |
| Colloidal silicon dioxide (Cab-O-Sil M-5 P) | 5.00 |
| Microcrystalline Cellulose (Avicel PH102) | 148.5 |
| Crospovidone (Polyplasdone XL) | 30.0 |
| Magnesium stearate | 5.00 |
| Total | 500.0 |

No sticking was observed during compression. By XRPD and DSC, the mannitol in the spray dried powder exists as δ form. The particle size recovery of the resulting spray dried powder was complete.

### EXAMPLE 5

### According to the Invention

### Composition 6

The following composition was prepared in a similar manner to Composition 3 in Example 2. The homogenous suspension was passed through a Netzsch bead mill (containing 85% by volume of yttrium-stabilised 0.3 mm zirconium oxide beads). The dispersion was re-circulated through the bead mill with continual mixing until a target particle size Dv90 of about 0.45 μm was obtained.

| Ingredients | Unit Formula % w/w |
|---|---|
| Talnetant, mioronised | 20.0 |
| Sodium Lauryl Sulfate | 0.300 |
| Povidone (Kollidon 30) | 5.00 |
| Mannitol Powder (Mannitol 60) | 10.0 |
| Purified Water | 64.7 |
| Total | 100.0 |
| Ratio of povidone:mannitol | 0.5 |
| Ratio of (povidone + mannitol):talnetant | 0.75 |

The composition was spray dried in a similar manner to Example 1. Spray drying was conducted on a Niro Mobile Minor spray dryer at the inlet/outlet temperatures of about 150°/60° C. The spray dried powder was then mixed with further excipients as follows:

| Ingredients | Unit formula (mg/tablet) |
|---|---|
| Talnetant spray dried powder, 56.7% w/w | 353 |
| consisting of: | |
| Talnetant, micronised | 200.0 |
| Sodium Lauryl Sulfate | 3.00 |
| Povidone (Kollidon 30) | 50.0 |
| Mannitol | 100.0 |
| Colloidal silicon dioxide (Cab-O-Sil M-5 P) | 5.00 |
| Microcrystalline Cellulose (Avicel PH102) | 107 |
| Crospovidone (Polyplasdone XL) | 30.0 |
| Magnesium stearate | 5.00 |
| Total | 500.0 |

No sticking was observed during compression. By XRPD and DSC, the mannitol in the spray dried powder exists as δ form. The particle size recovery of the resulting spray dried powder was complete.

1. A pharmaceutical composition comprising (i) talnetant, (ii) povidone, (iii) mannitol and (iv) a surfactant, wherein:
(a) the ratio of povidone to mannitol is 0.45:1 or higher; and
(b) the ratio of (povidone+mannitol) to talnetant is 0.3:1 or higher.

2. A composition as claimed in claim 1, wherein the ratio of povidone to mannitol is between 0.45:1 and 1:1.

3. A composition as claimed in claim 2, wherein the ratio of povidone to mannitol is between 0.58:1 and 0.62:1.

4. A composition as claimed in claim 1, wherein the ratio of (mannitol+povidone) to talnetant is between 0.3:1 and 5:1.

5. A composition as claimed in claim 4, wherein the ratio of (mannitol+povidone) to talnetant is between 0.6:1 and 1:1.

6. A composition as claimed in claim 5, wherein the ratio of (mannitol+povidone) to talnetant is between 0.7:1 and 0.8:1.

7. A composition as claimed in claim 1, wherein the crystalline mannitol is in the δ-form.

8. A composition as claimed in claim 1, wherein talnetant is in the form of talnetant particles having a Dv90 in the range from 0.1 to 2.0 μm.

9. A composition as claimed in claim 1, wherein the surfactant is an ionic surfactant.

10. A composition as claimed in claim 9, wherein the surfactant is sodium lauryl sulfate or dioctyl sodium sulfosuccinate (docusate sodium).

11. A composition as claimed in claim 1, wherein the surfactant is a non-ionic surfactant.

12. A composition as claimed in claim 11, wherein the surfactant is POE polyoxypropylene glycol.

13. A composition as claimed in claim 1, which also comprises water.

14. A composition as claimed in claim 13 wherein the composition comprises 25% to 90% water by unit formula % w/w.

15. A composition as claimed in claim 14 which consists of:

| Ingredients | Unit Formula % w/w |
|---|---|
| Talnetant | 20.0 |
| Sodium Lauryl Sulfate | 0.3 |
| Povidone | 5.5 |
| Mannitol | 9.5 |
| Water | 64.7 |

16. A process for the preparation of a spray-dried composition, the process comprising:
(1) wet milling a dispersion of (i) talnetant, (ii) povidone, (iii) mannitol and (iv) a surfactant, wherein:
(a) the ratio of povidone to mannitol is 0.45:1 or higher; and
(b) the ratio of (povidone+mannitol) to talnetant is 0.3:1 or higher.
(2) spray drying or spray granulating the resulting dispersion.

17. A spray dried composition obtainable according to the process as defined in claim 16.

18. A dosage form comprising a composition as defined in claim 1.

19. A dosage form as claimed in claim 18 which is for oral administration.

20. A dosage form as claimed in claim 19 which is a tablet.

21. A dosage form as claimed in claim 1 which consists of:

| Ingredients | Unit Formula % w/w |
|---|---|
| Talnetant | 38.46 |
| Sodium Lauryl Sulfate | 0.58 |
| Povidone | 10.58 |
| Mannitol Powder | 18.27 |
| Excipient(s) | to make up 100% |

22. A dosage form as claimed in claim 21 which consists of:

| Ingredients | Unit Formula % w/w |
|---|---|
| Talnetant Spray Dried Powder | 67.9 |
| consisting of: | |
| Talnetant drug substance | 38.46 |
| Sodium Lauryl Sulfate | 0.58 |
| Povidone (Kollidon 30) | 10.58 |
| Mannitol | 18.27 |
| Microcrystalline Cellulose | 20.6 |
| Colloidal Silicon Dioxide | 0.5 |
| Crospovidone | 10.0 |
| Magnesium Stearate | 1.00 |
| Total | 100.0 |
